(19) United States
(12) Patent Application Publication
(10) Pub. No.: US 2022/0296799 A1
(43) Pub. Date: Sep. 22, 2022

(54) PERCUTANEOUS CATHETER

(71) Applicant: TERUMO KABUSHIKI KAISHA, Tokyo (JP)

(72) Inventor: Kenji Yokoyama, Shizuoka (JP)

(21) Appl. No.: 17/834,239

(22) Filed: Jun. 7, 2022

Related U.S. Application Data
(63) Continuation of application No. PCT/JP2021/006066, filed on Feb. 18, 2021.

(30) Foreign Application Priority Data
Feb. 25, 2020 (JP) 2020-029665

(51) Int. Cl. A61M 1/36; A61M 60/113; A61M 60/232; A61M 60/38

(52) U.S. Cl. CPC A61M 1/3659; A61M 60/113; A61M 60/232; A61M 60/38

(57) ABSTRACT

A percutaneous catheter includes an expansion portion, a shaft portion, and an intermediate portion. The expansion portion includes a first reinforcing body including wires braided so as to intersect with each other. The shaft portion includes a second reinforcing body including the wires braided so as to intersect with each other. The intermediate portion includes a third reinforcing body including the wires braided so as to intersect with each other. The third reinforcing body is configured to have a braiding angle as an inner angle in an axial direction among angles formed by the intersecting wires which is smaller than those of the first reinforcing body and the second reinforcing body.

### CROSS REFERENCE TO RELATED APPLICATIONS

This application is a continuation of PCT Application No. PCT/JP2021/006066, filed Feb. 18, 2021, based on and claiming priority to Japanese Application No. JP2020-029665, filed Feb. 25, 2020, both of which are incorporated herein by reference in their entirety.

### BACKGROUND OF THE INVENTION

The present invention relates to a percutaneous catheter.

Treatment by percutaneous cardiopulmonary support (PCPS) has been conventionally performed in order to perform cardiopulmonary resuscitation, circulatory support, and respiratory support in emergency treatment. The percutaneous cardiopulmonary support is a method of temporarily assisting and substituting for a cardiopulmonary function using an extracorporeal circulation device.

The extracorporeal circulation device includes an extracorporeal circulation circuit including a centrifugal pump, an oxygenator, a blood removal path, a blood supply path, and the like, and performs gas exchange on removed blood and supplies the blood to the blood supply path.

When blood circulation is performed in the circulation circuit, blood is circulated by the force of a pump driven by a motor. Consequently, a reduction in a pressure loss in a tube constituting the circulation circuit is required to preferably perform blood circulation.

However, when an inner diameter of the tube is small, the pressure loss increases, and a flow rate through the circulation circuit decreases. Accordingly, unless the inner diameter of the tube is set to a sufficient size, a required circulating amount of blood cannot be obtained.

On the other hand, when the inner diameter of the tube is increased, an outer diameter of the tube is also increased. Consequently, when an inner diameter of a blood removal catheter (tube) or a blood supply catheter (tube) inserted into a patient's body is increased, the degree of invasion of the patient's body increases, and a burden on the patient's body increases.

In relation to this, for example, U.S. Pat. No. 6,626,859 discloses a high-performance cannula capable of extending or contracting a cannula body (e.g., catheter) in an axial direction by a mandrel (e.g., stylet or dilator) to increase or decrease a diameter. According to the high-performance cannula configured as described above, by inserting the cannula body into a living body in a state where the cannula body is extended in the axial direction and the diameter (outer diameter) is reduced by being stretched over the mandrel, the degree of invasion of a patient's body is reduced. Further, by removing the mandrel after inserting the high-performance cannula into the living body, the cannula body contracts in the axial direction to increase the diameter (inner diameter). Accordingly, a pressure loss in the catheter is reduced, and a required flow rate of liquid can be secured.

In the high-performance cannula disclosed in U.S. Pat. No. 6,626,859, when the stylet is inserted, there is a possibility that the stylet becomes tightly bound to the cannula (i.e., pinched by the cannula) in the vicinity of an insertion point between a proximal end and a distal end so that the inserted stylet can no longer move (e.g., slide) relative to the cannula. This constriction of motion may impede the desired stretching of the cannula.

### SUMMARY OF THE INVENTION

The present invention has been made to solve the above problems, and an object of the present invention is to provide a percutaneous catheter capable of suppressing a burden on a patient's body, reducing a pressure loss of liquid during circulation through a circulation circuit to secure a required flow rate of liquid, and preferably preventing tightening of a stylet when the stylet is inserted.

A percutaneous catheter that achieves the above object extends in an axial direction and allows blood to pass therethrough. The percutaneous catheter includes an expansion portion extending in the axial direction to be expandable, a shaft portion extending in the axial direction and provided on a proximal end side in an insertion direction of the expansion portion, and an intermediate portion provided between the expansion portion and the shaft portion. The expansion portion has an inner and outer diameter larger than those of the shaft portion and is configured to have higher elasticity than that of the shaft portion. The intermediate portion is configured to have an inner and outer diameter gradually decreasing from the expansion portion toward the shaft portion. The expansion portion includes a first reinforcing body including wires which are braided so as to intersect with each other, the shaft portion includes a second reinforcing body including wires which are braided so as to intersect with each other, and the intermediate portion includes a third reinforcing body including wires which are braided so as to intersect with each other. The wires may be wound continuously from one portion to the next, with the different characteristics. The third reinforcing body (in the intermediate portion) is configured to have a braiding angle as an inner angle in the axial direction among angles formed by the intersecting wires which is smaller than those of the first reinforcing body and the second reinforcing body. The smaller braiding angle results in a proportionally smaller amount of decrease in the diameter of the intermediate portion during axial expansion.

In one aspect of the invention, a percutaneous catheter comprises an elongated tubular body with (A) an expansion portion extending in the axial direction to be expandable and having a respective inner diameter and a respective outer diameter, (B) a shaft portion extending in the axial direction and provided on a proximal end side in an insertion direction of the expansion portion and having a respective inner diameter and a respective outer diameter, and (C) an intermediate portion provided between the expansion portion and the shaft portion. The respective inner diameter and outer diameter of the expansion portion are respectively larger than the inner diameter and outer diameter of the shaft portion, and the expansion portion is configured to have higher elasticity than that of the shaft portion. The intermediate portion is configured to have a respective inner diameter and a respective outer diameter gradually decreasing from the expansion portion to the shaft portion. The expansion portion comprises a first reinforcing body including first braided wires which intersect with each other in the first reinforcing body at a first braiding angle. The shaft portion comprises a second reinforcing body including second braided wires which intersect with each other at a second braiding angle. The intermediate portion includes a third reinforcing body including third braided wires which intersect with each other at a third braiding angle. The third reinforcing body is configured such that the third braiding angle is smaller than the first braiding angle and the second braiding angle.

In another aspect of the invention, the third braiding angle may vary along at different axial positions on the third reinforcing body. There may be a first region in which the third braiding angle is configured to gradually decrease when moving away from the first braiding angle of the first reinforcing body, and a second region in which the third braiding angle is configured to gradually increase continuously from the final braiding angle at an end of the first region toward the second braiding angle of the second reinforcing body.

According to the percutaneous catheter configured as described above, since the percutaneous catheter is inserted into a living body in a state where the expansion portion extends in the axial direction and the outer diameter is reduced, the burden on the patient's body can be suppressed. Additionally, when the stylet is removed from the percutaneous catheter after the percutaneous catheter is placed in the living body, the expansion portion contracts in the axial direction and returns to the original state. Here, since the expansion portion has the inner diameter larger than that of the shaft portion, a pressure loss in the expansion portion is reduced and a required flow rate of liquid can be secured.

When the expansion portion and the intermediate portion extend in the axial direction, the wires constituting the first reinforcing body of the expansion portion and the third reinforcing body of the intermediate portion are deformed such that an inclination angle of the wire braiding with respect to the axial direction gradually decreases. Here, since the third reinforcing body is configured to have the braiding angle as the inner angle in the axial direction among the angles formed by the intersecting wires which is smaller than those of the first reinforcing body and the second reinforcing body, the inclination angle of the wires constituting the third reinforcing body with respect to the axial direction is smaller, whereby an expansion (stretching) distance along the axial direction of the intermediate portion accompanying insertion of the stylet into the percutaneous catheter becomes relatively shorter than is the case where the braiding angle is larger. In this way, since the increase in the extension distance along the axial direction of the intermediate portion accompanying the insertion of the stylet into the percutaneous catheter is shortened, radially inward contraction of the intermediate portion is suppressed so that the inner diameter remains larger than a diameter of the stylet passing through the intermediate portion, and the stylet can be preferably prevented from being tightened against the catheter.

Therefore, it is possible to provide a percutaneous catheter capable of suppressing a burden on a patient's body, reducing a pressure loss of liquid during circulation through a circulation circuit to secure a required flow rate of liquid, and preferably preventing tightening of a stylet when the stylet is inserted.

### BRIEF DESCRIPTION OF DRAWINGS

FIG. 1 is a system diagram illustrating an example of an extracorporeal circulation device to which a percutaneous catheter according to an embodiment of the present invention is applied.

FIG. 2 is a side view illustrating a state before a stylet is inserted into a catheter according to a first embodiment.

FIG. 3 is a side cross-sectional view illustrating the catheter according to the first embodiment.

FIG. 4 is a side view illustrating a state after the stylet is inserted into the catheter.

FIG. 5A is a view for explaining a braiding angle of a first reinforcing body, FIG. 5B is a view for explaining a braiding angle of a second reinforcing body, and FIG. 5C is a view for explaining a braiding angle of a third reinforcing body.

FIG. 6 is a graph for explaining a braiding angle in an intermediate portion.

FIG. 7 is a side cross-sectional view illustrating the vicinity of the intermediate portion of the catheter.

FIG. 8 is a photograph showing a state when a stylet is inserted into a percutaneous catheter according to a comparative example.

FIG. 9 is a photograph showing a state when a stylet is inserted into a percutaneous catheter according to the first embodiment.

FIG. 10 is a plan view illustrating a state before a stylet is inserted into a catheter according to a second embodiment.

FIG. 11 is a side cross-sectional view illustrating the catheter according to the second embodiment.

FIG. 12 is a plan view illustrating a state after the stylet is inserted into the catheter according to the second embodiment.

FIG. 13 is a graph for explaining a braiding angle in an intermediate portion according to a modification.

### DETAILED DESCRIPTION OF PREFERRED EMBODIMENTS

Hereinafter, embodiments of the present invention will be described with reference to the accompanying drawings. Note that the following description does not limit the technical scope or meaning of terms described in the claims. Further, dimensional ratios in the drawings are exaggerated for convenience of description and may be different from actual ratios.

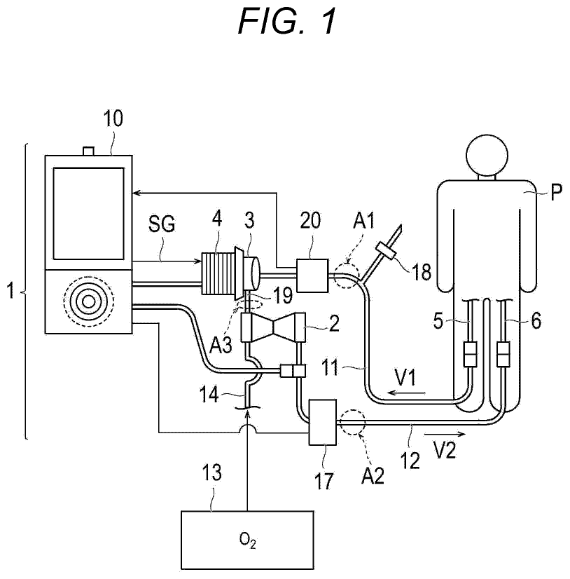

FIG. 1 is a system diagram illustrating an example of an extracorporeal circulation device to which a percutaneous catheter according to an embodiment of the present invention is applied and which is used as PCPS for temporarily assisting and substituting for functions of a heart and a lung until a heart function recovers when the heart of a patient is weak.

According to an extracorporeal circulation device 1, it is possible to perform a veno-arterial (VA) procedure of removing blood from a vein (vena cava) of a patient by operating a pump, exchanging gas in the blood by an oxygenator to oxygenate the blood, and then returning the blood to an artery (aorta) of the patient. The extracorporeal circulation device 1 is a device that assists a heart and a lung. Hereinafter, a procedure of removing blood from a patient, performing a predetermined treatment outside a body, and then supplying the blood into the patient's body again is referred to as “extracorporeal circulation”.

As illustrated in FIG. 1, the extracorporeal circulation device 1 includes a circulation circuit that circulates blood. The circulation circuit includes an oxygenator 2, a centrifugal pump 3, a drive motor 4 which is drive means for driving the centrifugal pump 3, a venous side catheter (percutaneous catheter for blood removal) 5, an arterial side catheter (catheter for blood supply) 6, and a controller 10 as a control unit.

The venous side catheter (catheter for blood removal) 5 is inserted from the femoral vein, and a distal end of the venous side catheter 5 is placed in the right atrium via the inferior vena cava. The venous side catheter 5 is connected to the centrifugal pump 3 via a blood removal tube (blood removal line) 11. The blood removal tube 11 is a conduit for sending blood.

The arterial side catheter (catheter for blood supply) 6 is inserted from the femoral artery.

When the drive motor 4 operates the centrifugal pump 3 according to a command SG of the controller 10, the centrifugal pump 3 can return blood to a patient P via a blood supply tube (blood supply line) 12 after removing the blood from the blood removal tube 11 and passing the blood through the oxygenator 2.

The oxygenator 2 is arranged between the centrifugal pump 3 and the blood supply tube 12. The oxygenator 2 performs gas exchange (oxygenation and/or carbon dioxide removal) on the blood. The oxygenator 2 is, for example, a membrane oxygenator, and a hollow fiber membrane oxygenator is particularly preferably used. Oxygen gas is supplied from an oxygen gas supply unit 13 to the oxygenator 2 through a tube 14. The blood supply tube 12 is a conduit connecting the oxygenator 2 and the arterial side catheter 6.

As the blood removal tube 11 and the blood supply tube 12, for example, a conduit made of a synthetic resin having high transparency and elastically deformable flexibility such as a vinyl chloride resin or a silicone rubber can be used. In the blood removal tube 11, the blood as liquid flows in the V1 direction, and in the blood supply tube 12, the blood flows in the V2 direction.

In the circulation circuit illustrated in FIG. 1, an ultrasonic bubble detection sensor 20 is arranged in the middle of the blood removal tube 11. A fast clamp 17 is arranged in the middle of the blood supply tube 12.

When air bubbles are mixed in the circulation circuit due to an erroneous operation of a three-way stopcock 18, breakage of the tube, or the like during extracorporeal circulation, the ultrasonic bubble detection sensor 20 detects the mixed air bubbles. When the ultrasonic bubble detection sensor 20 detects that there are air bubbles in the blood sent into the blood removal tube 11, the ultrasonic bubble detection sensor 20 sends a detection signal to the controller 10. Based on this detection signal, the controller 10 gives a warning by an alarm, and decreases the rotation speed of the centrifugal pump 3 or stops the centrifugal pump 3. Further, the controller 10 gives a command to the fast clamp 17, and the fast clamp 17 immediately closes the blood supply tube 12. This prevents air bubbles from being sent into a body of the patient P. The controller 10 controls the operation of the extracorporeal circulation device 1 to prevent air bubbles from mixing into the body of the patient P.

The tube 11 (12, 19) of the circulation circuit of the extracorporeal circulation device 1 is provided with a pressure sensor. For example, the pressure sensor can be installed in any one or all of the installation position A1 of the blood removal tube 11, the installation position A2 of the blood supply tube 12 of the circulation circuit, and the installation position A3 of a connection tube 19 connecting the centrifugal pump 3 and the oxygenator 2. In this way, a pressure in the tube 11 (12, 19) can be measured by the pressure sensor when the extracorporeal circulation is performed on the patient P by the extracorporeal circulation device 1. The installation position of the pressure sensor is not limited to the installation positions A1, A2, and A3, and the pressure sensor can be installed in any position of the circulation circuit.

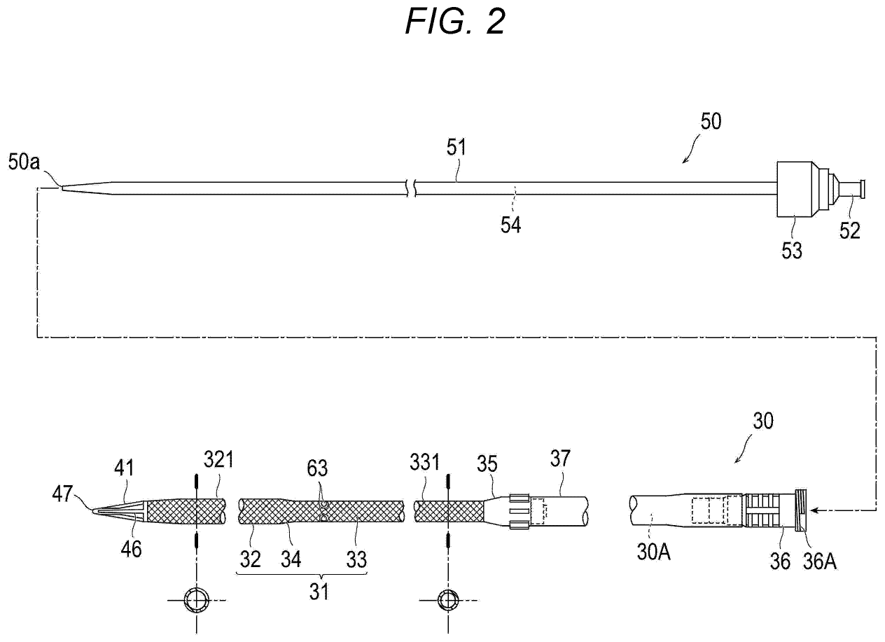

A percutaneous catheter (hereinafter, may be referred to as “catheter”) 30 according to a first embodiment of the present invention will be described with reference to FIGS. 2 to 7. FIGS. 2 to 7 are views for explaining a configuration of the catheter 30 according to the first embodiment. The catheter 30 is used as the venous side catheter (catheter for blood removal) 5 in FIG. 1.

As illustrated in FIG. 2, the catheter 30 according to the present embodiment includes a catheter tube 31 having side holes 63, a distal end tip 41 arranged at a distal end of the catheter tube 31 and having through holes 46 and 47, a clamping tube 37 arranged on a proximal end side of the catheter tube 31, a catheter connector 35 connecting the catheter tube 31 and the clamping tube 37, and a lock connector 36.

Note that a side to be inserted into a living body is referred to as “distal end” or “distal end side”, and a hand side where an operator performs an operation is referred to as “proximal end” or “proximal end side” in the present specification. A distal end portion means a certain range including the distal end (most distal end) and its periphery, and a proximal end portion means a certain range including the proximal end (most proximal end) and its periphery.

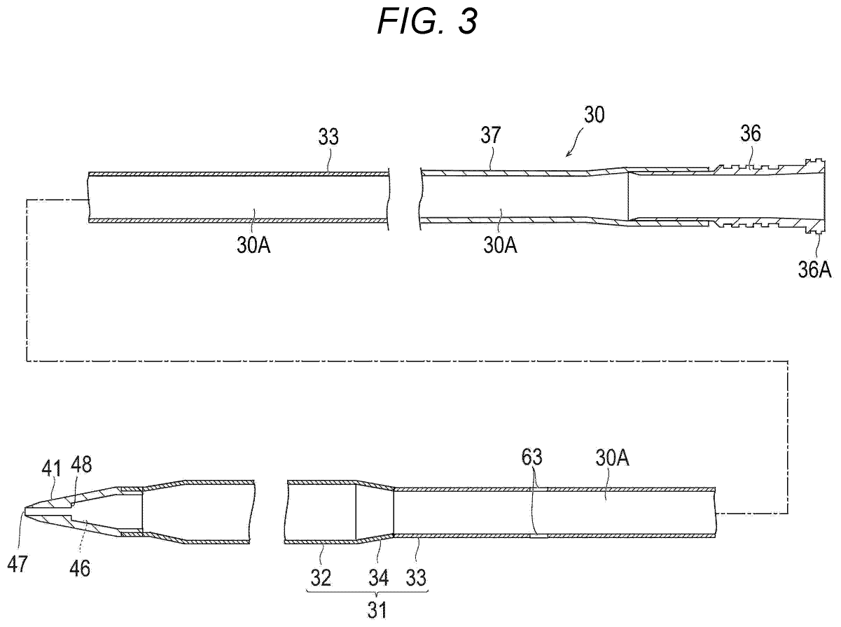

As illustrated in FIG. 3, the catheter 30 has a lumen 30A penetrating from the distal end to the proximal end. The through holes 46 and 47 included in the distal end tip 41 and the side holes 63 are arranged in different blood removal targets in the living body and configured to efficiently remove blood.

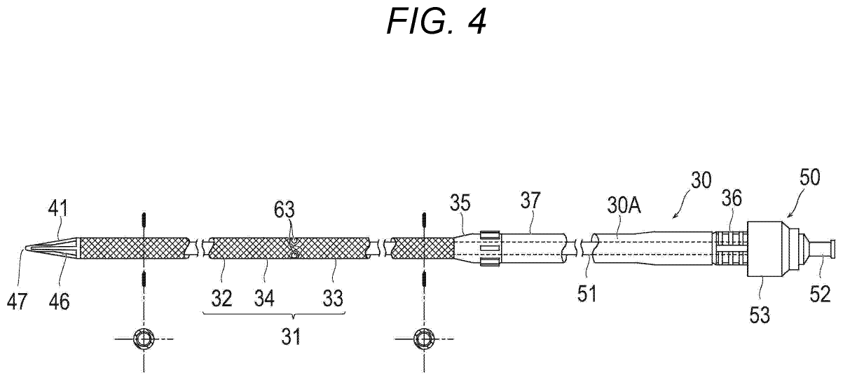

When the catheter 30 is inserted into the living body, a stylet 50 illustrated in FIG. 2 is used. As illustrated in FIG. 4, the stylet 50 is inserted into the lumen 30A of the catheter 30, and the catheter 30 and the stylet 50 are inserted into the living body in a state of being integrated in advance. Note that a method of using the catheter 30 will be described later.

Hereinafter, each component of the catheter 30 will be described.

As illustrated in FIG. 2, the catheter tube 31 includes an expansion portion 32 that is expandable in the axial direction, a shaft portion 33 provided on a proximal end side of the expansion portion 32, and an intermediate portion 34 provided between the expansion portion 32 and the shaft portion 33 which is also expandable in the axial direction.

The expansion portion 32 and the intermediate portion 34 are configured to have higher elasticity than that of the shaft portion 33, although shaft portion 33 may also expandable in the axial direction. As illustrated in FIG. 3, the expansion portion 32 is configured to have an outer diameter and an inner diameter larger than those of the shaft portion 33. Further, as illustrated in FIG. 3, the intermediate portion 34 is configured to have an inner diameter and an outer diameter gradually decreasing from the expansion portion 32 to the shaft portion 33. In other words, the intermediate portion 34 is tapered such that the inner diameter and the outer diameter increase toward the distal end side. The shaft portion 33, intermediate portion 34, and expansion portion 32 may all have the same or similar radial thicknesses.

The expansion portion 32, the shaft portion 33, and the intermediate portion 34 are configured to have lengths necessary for arranging the through holes 46 and 47 of the distal end tip 41 and the side holes 63 in desired blood removal targets. The length of the expansion portion 32 can be, for example, 10 to 40 cm, the length of the shaft portion 33 can be, for example, 20 to 40 cm, and the length of the intermediate portion 34 can be 3 to 4 cm.

The side holes 63 are provided in the shaft portion 33. The side holes 63 function as blood removal holes. A plurality of the side holes 63 are preferably provided in a circumferential direction. In the present embodiment, four side holes 63 are provided in the circumferential direction. Accordingly, even when one side hole 63 is adsorbed to a blood vessel wall and blocked due to blood removal, the blood removal can be performed through another side hole 63, so that blood circulation can be stably performed.

In the present embodiment, the blood removal targets are two places of the right atrium and the inferior vena cava. The catheter 30 is inserted into and placed in the living body such that the through holes 46 and 47 of the distal end tip 41 are arrange in the right atrium and the side holes 63 are arranged in the inferior vena cava.

In a state where the through holes 46 and 47 and the side holes 63 are arranged in the blood removal targets, the expansion portion 32 is arranged in the inferior vena cava, which is a relatively thick blood vessel, and the shaft portion 33 is arranged in the femoral vein, which is a relatively thin blood vessel.

Additionally, when the stylet 50 is inserted into the lumen 30A of the catheter 30 to press against and axially displace distal end tip 41, the expansion portion 32 and the intermediate portion 34 having high elasticity extend (i.e., axially expand or stretch) in an axial direction and the outer diameters and the inner diameters decrease as illustrated in FIG. 4. As a result, the outer diameters of the expansion portion 32 and the intermediate portion 34 become approximately the same as the outer diameter of the shaft portion 33. Since the catheter 30 is inserted into the living body in a state where the expansion portion 32 and the intermediate portion 34 extend in the axial direction and the outer diameters and the inner diameters are in a decreased state, the catheter 30 can be inserted in a minimally invasive manner.

Further, when the stylet 50 is removed from the lumen 30A of the catheter 30 after the catheter 30 is placed in the living body, the expansion portion 32 and the intermediate portion 34 contract from the state of extending in the axial direction so that the inner diameters increase. Here, the expansion portion 32 is arranged in the inferior vena cava, which is a relatively thick blood vessel. Consequently, the outer diameter of the expansion portion 32 can be increased, and the inner diameter can be increased accordingly.

Here, the pressure loss in the expansion portion 32 is the total length of the expansion portion 32 multiplied by the (average) passage sectional area. In other words, by increasing the inner diameter of the expansion portion 32, the pressure loss in the expansion portion 32 is reduced. When the pressure loss in the expansion portion 32 is reduced, a flow rate of blood flowing through a circulation circuit increases. Accordingly, it is necessary to increase the inner diameter of the expansion portion 32 to obtain a sufficient circulating amount of blood.

On the other hand, in a case where wall thicknesses are approximately constant, when the inner diameters of the expansion portion 32, the shaft portion 33, and the intermediate portion 34 are increased, the outer diameters increase, and thus a burden on a patient is increased when the catheter 30 is inserted into the living body, which hinders a minimally invasive procedure.

From the above viewpoint, the inner diameter of the expansion portion 32 can be, for example, 9 to 11 mm, and the inner diameter of the shaft portion 33 can be, for example, 4 to 8 mm. Further, the radial wall thicknesses of the expansion portion 32, the shaft portion 33, and the intermediate portion 34 can be, for example, 0.4 to 0.5 mm.

Additionally, as illustrated in FIG. 2, it is preferable that a distal end portion of the expansion portion 32 form a tapered portion that gradually narrows from the center of the expansion portion 32 toward the outside in the axial direction. Accordingly, the inner diameter of the distal end of the expansion portion 32 is continuous with an inner diameter of the distal end tip 41 arranged on the distal end side.

Hereinafter, configurations of the expansion portion 32, the shaft portion 33, and the intermediate portion 34 will be described in more detail.

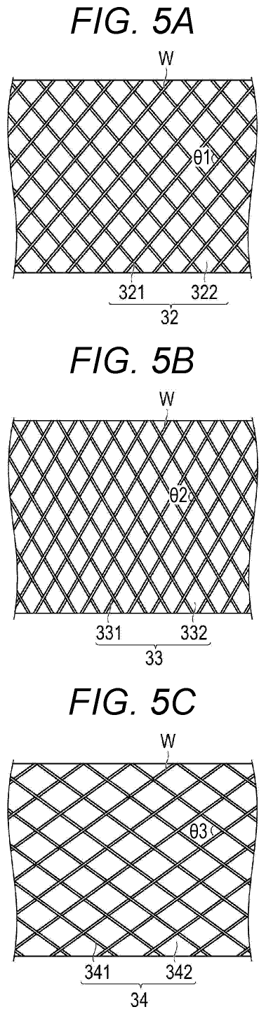

As illustrated in FIG. 5A, the expansion portion 32 includes a first reinforcing body 321 including wires W braided so as to intersect with each other, and a first resin layer 322 provided so as to cover the first reinforcing body 321.

As illustrated in FIG. 5B, the shaft portion 33 includes a second reinforcing body 331 including the wires W braided so as to intersect with each other, and a second resin layer 332 provided so as to cover the second reinforcing body 331.

As illustrated in FIG. 5C, the intermediate portion 34 includes a third reinforcing body 341 including the wires W braided so as to intersect with each other, and a third resin layer 342 provided so as to cover the third reinforcing body 341.

As illustrated in FIG. 5A, the first reinforcing body 321 is configured by braiding the wires W so as to have a first braiding angle θ1. Further, as illustrated in FIG. 5B, the second reinforcing body 331 is configured by braiding the wires W so as to have a second braiding angle θ2. Additionally, as illustrated in FIG. 5C, the third reinforcing body 341 is configured by braiding the wires W so as to have a third braiding angle θ3.

In the present specification, the braiding angles θ1, θ2, and θ3 are defined as inner angles in the axial direction among angles formed by the intersecting wires W, as illustrated in FIGS. 5A, 5B, and 5C.

As illustrated in FIGS. 5A and 5B, the first braiding angle θ1 of the first reinforcing body 321 is configured to be smaller than the second braiding angle θ2 of the second reinforcing body 331. Accordingly, an inclination angle of the wire W constituting the first reinforcing body 321 with respect to the axial direction is smaller as compared with a case where a braiding angle of the first reinforcing body 321 is larger than a braiding angle of the second reinforcing body 331.

Here, as the expansion portion 32 extends in the axial direction, the wire W constituting the first reinforcing body 321 of the expansion portion 32 is deformed such that the inclination angle with respect to the axial direction gradually decreases in response to the axial stretching of expansion portion 32. Then, when the inclination angle of the wire W constituting the first reinforcing body 321 of the expansion portion 32 with respect to the axial direction becomes approximately 0, the extension of the expansion portion 32 in the axial direction is halted.

Therefore, by configuring the first braiding angle θ1 of the first reinforcing body 321 to be smaller than the braiding angle θ2 of the second reinforcing body 331, as compared with the case where the braiding angle of the first reinforcing body 321 is larger than the braiding angle of the second reinforcing body 331, an extension distance along the axial direction of the expansion portion 32 accompanying insertion of the stylet 50 into the catheter 30 is shortened (i.e., axial stretching of expansion portion 32 is halted before the stretching of shaft portion 33.

The braiding angle θ1 of the first reinforcing body 321 is not particularly limited, but is 100 degrees to 120 degrees. Further, the braiding angle θ2 of the second reinforcing body 331 is not particularly limited, but is 130 degrees to 150 degrees. Accordingly, the kink resistance of the second reinforcing body 331 can be improved by making the braiding angle θ2 of the second reinforcing body 331 larger than the braiding angle θ1 of the first reinforcing body 321. Consequently, the catheter 30 can be preferably inserted into the living body in the femoral vein having a complicated configuration.

As illustrated in FIGS. 5A and 5B, the first reinforcing body 321 of the expansion portion 32 is braided so as to be sparser than the second reinforcing body 331 of the shaft portion 33. According to this configuration, the expansion portion 32 can be softened as compared with the shaft portion 33, and elasticity can be enhanced.

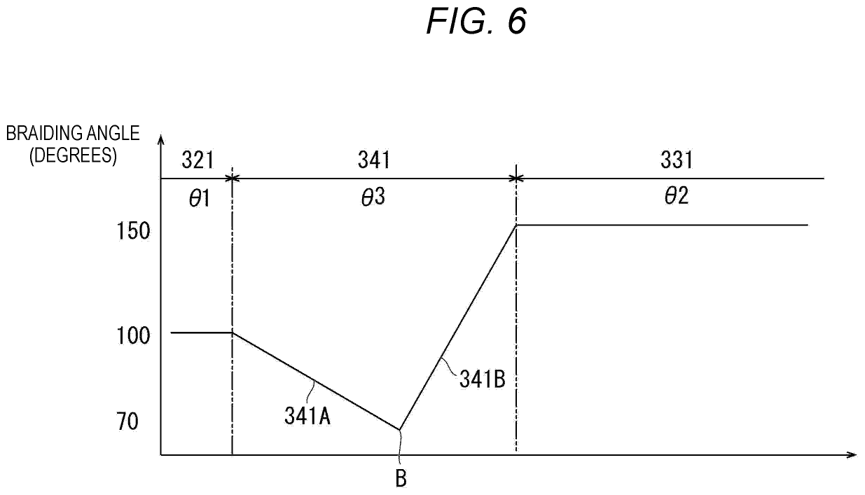

As illustrated in FIGS. 5 and 6, along at least a majority of the axial length of the third reinforcing body 341 of the intermediate portion 34, the third braiding angle θ3 is configured to be smaller than the first braiding angle θ1 of the first reinforcing body 321 and the second braiding angle θ2 of the second reinforcing body 331.

Specifically, as illustrated in FIG. 6, the third reinforcing body 341 has a first region 341A in which the third braiding angle θ3 is configured to gradually decrease from the first braiding angle θ1 of the first reinforcing body 321, and a second region 341B in which the third braiding angle θ3 is configured to gradually increase continuously from the first region 341A toward the second braiding angle θ2 of the second reinforcing body 331. The braiding angle θ3 of the third reinforcing body 341 at the boundary B between the first region 341A and the second region 341B is 50 degrees to 70 degrees.

When the intermediate portion 34 is expanded in the axial direction by insertion of the stylet 50, the braided wires W constituting the third reinforcing body 341 of the intermediate portion 34 are deformed such that the inclination angle with respect to the axial direction gradually decreases from its initial value. In this way, since the braiding angle θ3 of the third reinforcing body 341 is configured to be smaller than the braiding angle θ1 of the first reinforcing body 321 and the braiding angle θ2 of the second reinforcing body 331, as compared with a case where the braiding angle θ3 of the third reinforcing body 341 is equal to or larger than the braiding angle θ1 of the first reinforcing body 321 and the braiding angle θ2 of the second reinforcing body 331, the inclination angle of the wire W constituting the third reinforcing body 341 with respect to the axial direction becomes smaller sooner, and an extension distance along the axial direction of the intermediate portion 34 accompanying the insertion of the stylet 50 into the catheter 30 becomes shorter. Accordingly, since the extension distance along the axial direction of the intermediate portion 34 accompanying the insertion of the stylet 50 into the catheter 30 is shortened, radially inward contraction of the intermediate portion 34 is suppressed and the stylet 50 can be preferably prevented from being tightened.

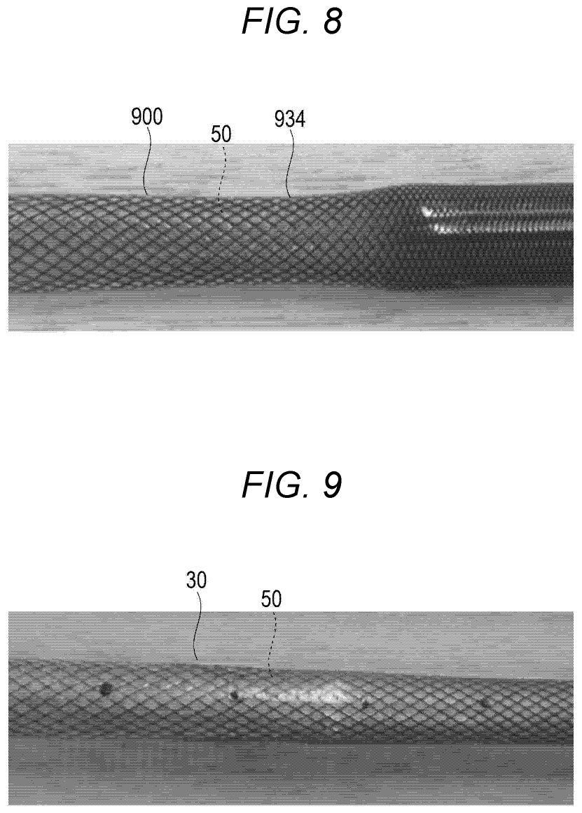

FIG. 8 is a photograph showing a state when the stylet 50 is inserted into a catheter 900 according to a comparative example. FIG. 9 is a photograph showing a state when the stylet 50 is inserted into the catheter 30 according to the present embodiment. In the catheter 900 according to the comparative example illustrated in FIG. 8, the braiding angle θ3 is configured to be larger than the braiding angle θ1 of the first reinforcing body 321. As illustrated in FIG. 8, by inserting the stylet 50 into the catheter 900, a lumen in an intermediate portion 934 of the catheter 900 contracts more radially inward than an outer diameter of the stylet 50, causing the stylet 50 to be undesirably tightened.

On the other hand, as illustrated in FIG. 9, according to the catheter 30 of the present embodiment, the radially inward contraction of the intermediate portion 34 is suppressed, and the stylet 50 can be preferably prevented from being tightened.

In the present embodiment, the wire W is made of a known shape memory material such as a shape memory metal or a shape memory resin. As the shape memory metal, for example, a titanium-based (Ni—Ti, Ti—Pd, Ti—Nb—Sn, etc.) or copper-based alloy can be used. Further, as the shape memory resin, for example, an acrylic resin, a trans-isoprene polymer, a polynorbornene, a styrene-butadiene copolymer, or polyurethane can be used.

Since the wire W is made of the shape memory material, a contraction distance along the axial direction of the expansion portion 32 accompanying removal of the stylet 50 from the catheter 30 is the same as the extension distance along the axial direction of the expansion portion 32 accompanying the insertion of the stylet 50 into the catheter 30.

A wire diameter of the wire W is preferably 0.1 mm to 0.2 mm.

Setting the wire diameter of the wire W to 0.1 mm or more can preferably exhibit a function as a reinforcing body for improving strength.

On the other hand, by setting the wire diameter of the wire W to 0.2 mm or less, the inner diameter can be increased while reducing the outer diameter of the expansion portion 32, so that it is possible to achieve both suppression of the burden on the patient's body at the time of inserting the catheter 30 and reduction in the pressure loss. Additionally, at this time, the wires W can be prevented from being exposed from the first resin layer 322 at a portion where the wires W are braided into two layers. In the present embodiment, a cross section of the wire W is circular, but is not limited thereto, and may be rectangular, square, elliptical, or the like.

The first resin layer 322 of the expansion portion 32 is made of a softer material having lower hardness than the second resin layer 332 of shaft portion 33. According to this configuration, the expansion portion 32 can be softened as compared with the shaft portion 33, and elasticity can be enhanced.

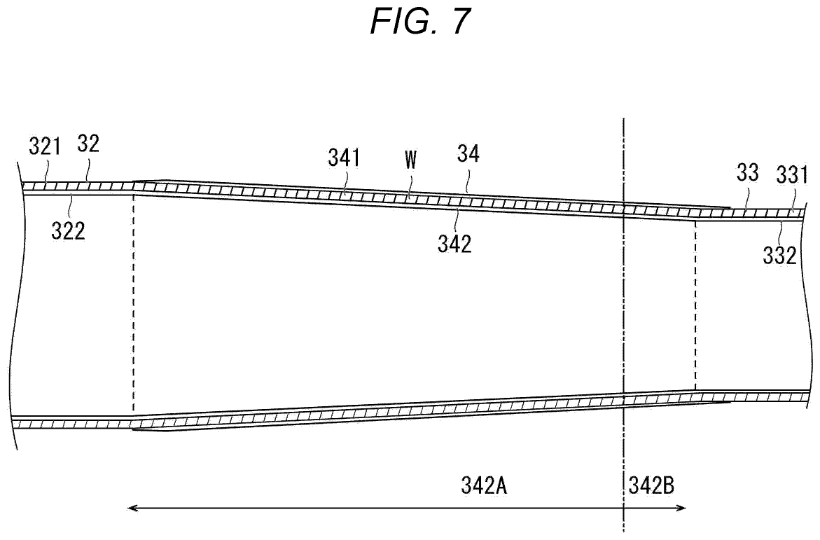

As illustrated in FIG. 7, the third resin layer 342 of the intermediate portion 34 has a first region 342A formed of the first resin layer 322 of the expansion portion 32, and a second region 342B formed of the first resin layer 322 of the expansion portion 32 and the second resin layer 332 of the shaft portion 33. A length of the second region 342B along the axial direction is not particularly limited, and is, for example, 5 to 8 mm.

The first and second resin layers 322 and 332 can be formed using vinyl chloride, silicon, polyethylene, nylon, urethane, polyurethane, a fluororesin, a thermoplastic elastomer resin, or the like, or using a composite material thereof.

Since the silicon material has high biocompatibility and the material itself is soft, it does not easily damage a blood vessel. The polyethylene material is soft and has hardness to withstand pressure. Moreover, the polyethylene material has biocompatibility comparable to that of the silicon material. The polyethylene material is harder than silicon and is easily inserted into a thin blood vessel. Additionally, the polyurethane material becomes soft after being inserted. As the materials of the first and second resin layers 322 and 332, applicable materials can be used by taking advantage of the features of these materials.

Further, a hydrophilic coating may be applied to the polyurethane material. In this case, a tube surface is smooth to be easily inserted into the blood vessel, and the blood vessel wall is not easily damaged. Blood and protein are less likely to adhere, and it can be expected that thrombus formation is prevented.

A method of forming the catheter tube 31 is not particularly limited, but the catheter tube can be formed by, for example, dip coating (dipping method) or insert molding. Note that it is sufficient that at least outer surfaces of the reinforcing bodies 321, 331, and 341 are covered with the resin layers 322, 332, and 342.

As illustrated in FIGS. 2 and 3, the distal end tip 41 is arranged at the distal end of the expansion portion 32. The distal end tip 41 has a tapered shape whose diameter is gradually reduced toward the distal end side.

As illustrated in FIG. 3, a flat receiving surface 48 that abuts on a flat surface 50a of the stylet 50 used before the catheter 30 is inserted into the living body is formed inside the distal end tip 41.

By fixing the hard distal end tip 41 to the distal end portion of the expansion portion 32, the expansion portion 32 can be effectively prevented from being crushed at the time of blood removal.

Note that a configuration of the distal end tip 41 is not limited to the configuration described above.

As illustrated in FIGS. 2 to 4, the clamping tube 37 is provided on a proximal end side of the shaft portion 33. A lumen into which the stylet 50 can be inserted is provided inside the clamping tube 37. The clamping tube 37 can be formed using the same material as that of the catheter tube 31.

As illustrated in FIGS. 2 and 4, the catheter connector 35 connects the shaft portion 33 and the clamping tube 37. A lumen into which the stylet 50 can be inserted is provided inside the catheter connector 35.

As illustrated in FIGS. 2 to 4, the lock connector 36 is connected to a proximal end side of the clamping tube 37. A lumen into which the stylet 50 can be inserted is provided inside the lock connector 36. A male screw portion 36A provided with a screw thread is provided on an outer surface on a proximal end side of the lock connector 36.

Next, a configuration of the stylet 50 will be described.

As illustrated in FIG. 2, the stylet 50 includes a stylet tube 51 provided extending in the axial direction, a stylet hub 52 to which a proximal end of the stylet tube 51 is fixed, and a screw ring 53 provided at a distal end of the stylet hub 52.

The stylet tube 51 is an elongated body extending in the axial direction and having relatively high rigidity. A total length along the axial direction of the stylet tube 51 is configured to be longer than a total length along the axial direction of the catheter 30. The stylet tube 51 includes a guide wire lumen 54 into which a guide wire (not illustrated) can be inserted. The stylet tube 51 is guided by the guide wire and inserted into the living body together with the catheter 30. After the catheter 30 is placed in the living body, the stylet tube 51 is removed from the catheter 30 by pulling out the stylet hub 52 to the proximal end side.

As illustrated in FIG. 2, a distal end of the stylet tube 51 includes the flat surface 50a on which the receiving surface 48 of the distal end tip 41 abuts. The stylet tube 51 has relatively high rigidity and stiffness that enables transmission of pushing force to the distal end side by operation at hand to the distal end tip 41. Accordingly, the stylet tube 51 expands a narrow blood vessel by bringing the flat surface 50a into contact with the receiving surface 48 of the distal end tip 41 and pushing the distal end tip 41 toward the distal end side.

The screw ring 53 includes a female screw portion (not illustrated) provided with a screw groove on an inner surface of the lumen. The stylet 50 is configured to be attachable to the catheter 30 by screwing the female screw portion of the screw ring 53 into the male screw portion 36A of the lock connector 36.

### <Method of Using the Catheter>

Next, a method of using the catheter 30 described above will be described. FIG. 2 illustrates a state before the stylet tube 51 of the stylet 50 is inserted into the lumen 30A of the catheter 30, and FIG. 4 illustrates a state after the stylet tube 51 is inserted into the lumen 30A of the catheter 30.

First, as illustrated in FIG. 4, the stylet tube 51 of the stylet 50 is inserted into the lumen 30A of the catheter 30. The stylet tube 51 sequentially passes through insides of the shaft portion 33 and the expansion portion 32, and the flat surface 50a of the stylet tube 51 abuts on the receiving surface 48 of the distal end tip 41.

Here, as illustrated in FIG. 2, the total length along the axial direction of the stylet tube 51 is configured to be longer than the total length along the axial direction of the catheter 30. Accordingly, the distal end tip 41 is pressed toward the distal end side in a state where the flat surface 50a of the stylet tube 51 abuts on the receiving surface 48 of the distal end tip 41. In this way, the distal end of the expansion portion 32 fixed to the distal end tip 41 is pulled toward the distal end side. Accordingly, the catheter 30 receives extending force in the axial direction, and the expansion portion 32 and the intermediate portion 34 that have relatively high elasticity in the catheter 30 extend in the axial direction. Subsequently, a proximal end of the catheter 30 is fixed to the stylet hub 52.

The expansion portion 32 extends in the axial direction, and the outer diameter of the expansion portion 32 decreases to be approximately the same as the outer diameter of the shaft portion 33 (see FIG. 4). As the expansion portion 32 extends in the axial direction, the wire W constituting the first reinforcing body 321 of the expansion portion 32 is deformed such that the inclination angle with respect to the axial direction gradually decreases. Further, as the intermediate portion 34 extends in the axial direction, the wire W constituting the third reinforcing body 341 of the intermediate portion 34 is deformed such that the inclination angle with respect to the axial direction gradually decreases.

As described above, since the catheter 30 according to the present embodiment is configured such that the third braiding angle θ3 of the third reinforcing body 341 is smaller than the first braiding angle θ1 of the first reinforcing body 321 and the second braiding angle θ2 of the second reinforcing body 331, as compared with a case where the third braiding angle θ3 of the third reinforcing body 341 is larger than the first braiding angle θ1 of the first reinforcing body 321 and the second braiding angle θ2 of the second reinforcing body 331, the inclination angle of the wire W constituting the third reinforcing body 341 with respect to the axial direction becomes small, and the extension distance along the axial direction of the intermediate portion 34 accompanying the insertion of the stylet 50 into the catheter 30 becomes short. Accordingly, since the extension distance along the axial direction of the intermediate portion 34 accompanying the insertion of the stylet 50 into the catheter 30 is shortened, radially inward contraction of the intermediate portion 34 is suppressed and the stylet 50 can be preferably prevented from being tightened.

Next, the catheter 30 into which the stylet 50 is inserted is inserted along the guide wire (not illustrated) previously inserted into a target site in the living body. At this time, since the stylet 50 is inserted into the catheter 30, the outer diameters of the expansion portion 32 and the intermediate portion 34 are approximately the same as the outer diameter of the shaft portion 33, and the catheter 30 can be inserted into the living body in a minimally invasive manner to suppress the burden on the patient's body.

In addition, the catheter 30 is inserted into and placed in the living body until the through holes 46 and 47 of the distal end tip 41 are arranged in the right atrium and the side holes 63 are arranged in the inferior vena cava. In a state where the through holes 46 and 47 and the side holes 63 are arranged in the blood removal targets, the expansion portion 32 is arranged in the inferior vena cava, which is a relatively thick blood vessel, and the shaft portion 33 is arranged in the femoral vein, which is a relatively thin blood vessel.

Next, the stylet tube 51 and the guide wire are removed from the catheter 30. At this time, the stylet tube 51 and the guide wire are once pulled out to a position of the clamping tube 37 of the catheter 30 and clamped by forceps (not illustrated), and then completely removed from the catheter 30. As the stylet tube 51 is removed from the lumen of the catheter 30, the catheter 30 is released from the extending force in the axial direction that the catheter 30 has received from the stylet 50. Consequently, the expansion portion 32 contracts in the axial direction, and the inner diameter of the expansion portion 32 increases. Accordingly, it is possible to reduce the pressure loss in the expansion portion 32 and secure a required flow rate of liquid.

Next, the lock connector 36 of the catheter 30 is connected to the blood removal tube 11 of the extracorporeal circulation device of FIG. 1. After confirming that the connection of the catheter on a blood supply side has been completed, the forceps of the clamping tube 37 are released to start extracorporeal circulation.

When the extracorporeal circulation is over, the catheter 30 is removed from the blood vessel and hemostatic repair is performed on an insertion site by a surgical procedure as necessary.

As described above, the catheter 30 according to the present embodiment extends in the axial direction and allows blood to pass therethrough. The catheter 30 includes the expansion portion 32 extending in the axial direction to be expandable, the shaft portion 33 extending in the axial direction and provided on the proximal end side in the insertion direction of the expansion portion 32, and the intermediate portion 34 provided between the expansion portion 32 and the shaft portion 33. The expansion portion 32 has an inner and outer diameter larger than that of the shaft portion 33, and is configured to have higher elasticity than that of the shaft portion 33. The intermediate portion 34 is configured to have the inner and outer diameter gradually decreasing from the expansion portion 32 toward the shaft portion 33. The expansion portion 32 includes the first reinforcing body 321 including the wires W braided so as to intersect with each other, the shaft portion 33 includes the second reinforcing body 331 including the wires W braided so as to intersect with each other, and the intermediate portion 34 includes the third reinforcing body 341 including the wires W braided so as to intersect with each other. The third reinforcing body 341 is configured to have a third braiding angle θ3 as the inner angle in the axial direction among the angles formed by the intersecting wires W which is smaller than those of the first reinforcing body 321 and the second reinforcing body 331.

According to the catheter 30 configured as described above, since the catheter 30 is inserted into the living body in a state where the expansion portion 32 extends in the axial direction and the outer diameter is reduced, the burden on the patient's body can be suppressed. Additionally, when the stylet 50 is removed from the catheter 30 after the catheter 30 is placed in the living body, the expansion portion 32 contracts in the axial direction and returns to the original state. Here, since the expansion portion 32 has the inner diameter larger than that of the shaft portion 33, the pressure loss in the expansion portion 32 is reduced and a required flow rate of liquid can be secured.

Further, when the expansion portion 32 and the intermediate portion 34 extend in the axial direction, the wires W constituting the first reinforcing body 321 of the expansion portion 32 and the third reinforcing body 341 of the intermediate portion 34 are deformed such that the inclination angle with respect to the axial direction gradually decreases. Here, since the third reinforcing body 341 is configured to have the third braiding angle θ3 as the inner angle in the axial direction among the angles formed by the intersecting wires W which is smaller than those of the first reinforcing body 321 and the second reinforcing body 331, the inclination angle of the wire W constituting the third reinforcing body with respect to the axial direction becomes small and the extension distance along the axial direction of the intermediate portion 34 accompanying the insertion of the stylet 50 into the catheter 30 becomes short, as compared with a case where the third braiding angle of the third reinforcing body is larger than the braiding angles of the first reinforcing body and the second reinforcing body. Accordingly, since the extension distance along the axial direction of the intermediate portion 34 accompanying the insertion of the stylet 50 into the catheter 30 is shortened, radially inward contraction of the intermediate portion 34 is suppressed and the stylet 50 can be preferably prevented from being tightened.

Therefore, it is possible to provide the catheter 30 capable of reducing the pressure loss of liquid during circulation through the circulation circuit to secure a required flow rate of liquid without increasing invasion of and the burden on the patient's body, and preferably preventing tightening of the stylet 50 when the stylet 50 is inserted.

Further, the third reinforcing body 341 may be provided with a first region 341A in which the third braiding angle θ3 is configured to gradually decrease from the first braiding angle θ1 of the first reinforcing body 321, and the second region 341B in which the third braiding angle θ3 is configured to gradually increase continuously from the first region 341A toward the second braiding angle θ2 of the second reinforcing body 331. According to the catheter 30 configured as described above, the length along the axial direction of the intermediate portion 34 can be shortened, and the catheter 30 can also be preferably applied to a catheter having a relatively short length along the axial direction.

### Second Embodiment

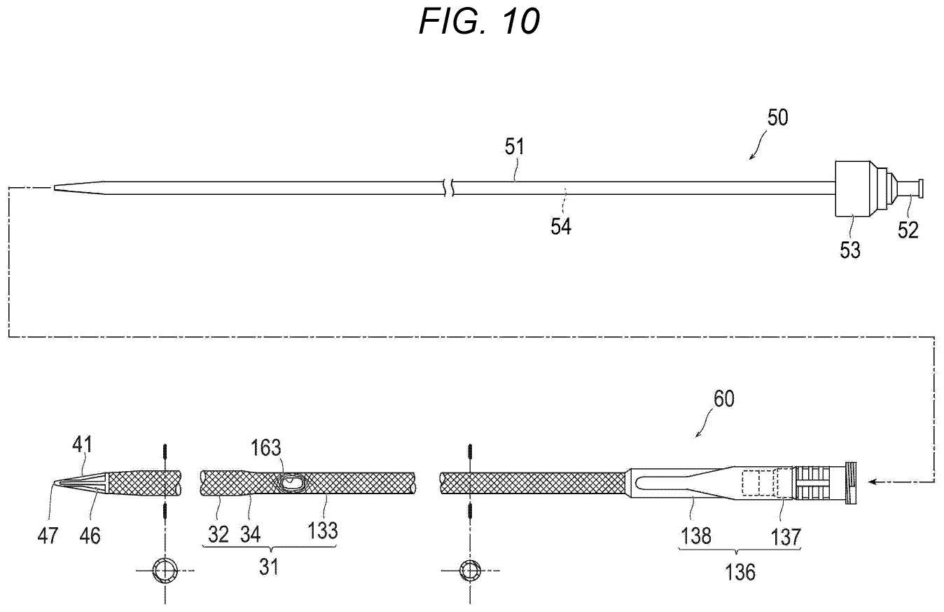

A percutaneous catheter (hereinafter, referred to as “catheter”) 60 according to a second embodiment of the present invention will be described with reference to FIGS. 10 to 12. FIGS. 10 to 12 are views for explaining a configuration of the catheter 60 according to the second embodiment.

The catheter 60 is a so-called double lumen catheter, and can simultaneously perform both blood supply and blood removal. Consequently, in the present embodiment, a procedure is performed using only one catheter 60 without using two catheters of a venous side catheter (catheter for blood removal) 5 and an arterial side catheter (catheter for blood supply) 6 in the extracorporeal circulation device of FIG. 1.

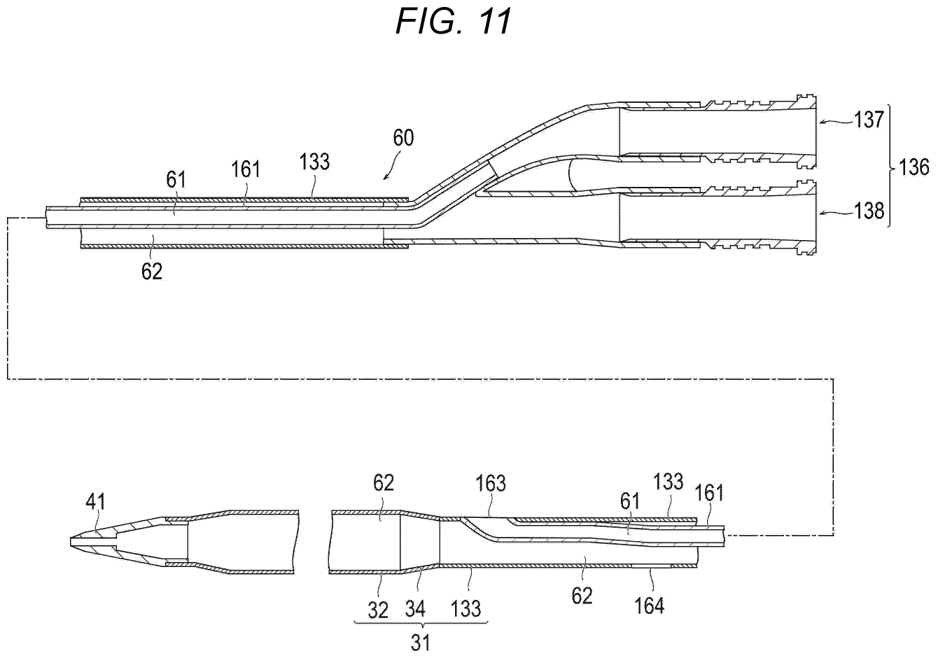

As illustrated in FIGS. 10 and 11, the catheter 60 according to the second embodiment is different from the catheter 30 according to the first embodiment in that a third tube 161 including a first lumen 61 communicating with a blood supply side hole 163 has a double tube structure arranged in a lumen of a shaft portion 133.

According to the catheter 60 according to the second embodiment, it is possible to perform veno-venous (VV) oxygenator extracorporeal blood circulation of removing blood from a vein (vena cava) of a patient by operating a pump of an extracorporeal circulation device, exchanging gas in the blood by an oxygenator to oxygenate the blood, and then returning the blood to the vein (vena cava) of the patient.

Hereinafter, each component of the catheter 60 will be described. Note that descriptions of parts common with the first embodiment will be omitted, and parts having features only in the second embodiment will be described. Further, the same parts as those of the first embodiment described above will be denoted by the same reference numerals to be described, and overlapping descriptions will be omitted.

As illustrated in FIGS. 10 to 12, the catheter 60 includes an expansion portion 32, a shaft portion 133, an intermediate portion 34, a distal end tip 41 arranged at a distal end of the expansion portion 32, and a third tube 161 arranged in a lumen of the shaft portion 133. Since the configurations of the expansion portion 32, the intermediate portion 34, and the distal end tip 41 are the same as those of the catheter 30 of the first embodiment, descriptions of thereof will be omitted.

As illustrated in FIG. 11, the catheter 60 includes the first lumen 61 functioning as a blood supply path and a second lumen 62 functioning as a blood removal path.

The first lumen 61 is formed in a lumen of the third tube 161. The second lumen 62 is formed in the lumens of the expansion portion 32, the intermediate portion 34, and the shaft portion 133, and penetrates from a distal end to a proximal end.

The shaft portion 133 is provided with the blood supply side hole 163 communicating with the first lumen 61 as a blood supply path.

The shaft portion 133 includes a blood removal side hole 164 communicating with the second lumen 62 as a blood removal path.

The blood supply side hole 163 and the blood removal side hole 164 are formed in an elliptical shape.

The third tube 161 is inserted into the second lumen 62 from a proximal end side of the shaft portion 133 and connected to the blood supply side hole 163.

The blood supply side hole 163 is arranged in a blood supply target in a living body, and blood oxygenated by the oxygenator is fed into the living body through the blood supply side hole 163.

Through holes 46 and 47 included in the distal end tip 41 and the blood removal side hole 164 included in the shaft portion 133 are arranged in different blood removal targets in the living body and configured to effectively remove blood. Further, even when the through holes 46 and 47 or the blood removal side hole 164 is adsorbed to a blood vessel wall and blocked, blood removal can be performed through the unblocked hole, so that extracorporeal circulation can be stably performed.

In the present embodiment, the catheter 60 is inserted from the internal jugular vein of a neck, and a distal end is placed in the inferior vena cava via the superior vena cava and the right atrium. The blood supply target is the right atrium, and the blood removal targets are two places of the superior vena cava and the inferior vena cava.

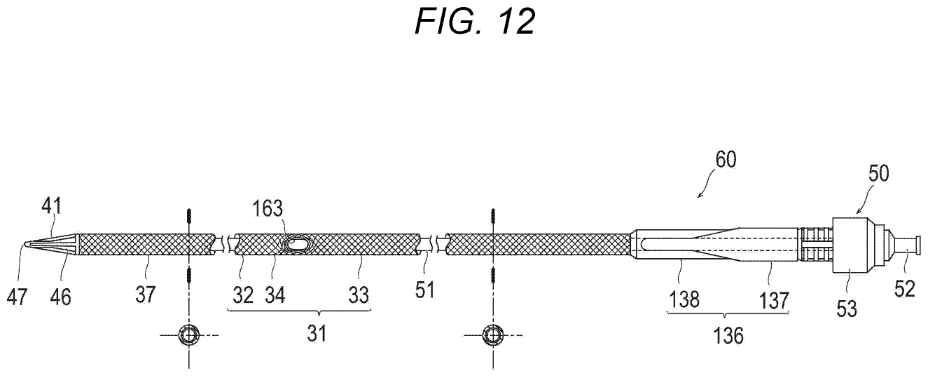

As illustrated in FIG. 12, in a state where a stylet 50 is inserted, the catheter 60 is inserted into and placed in the living body such that the through holes 46 and 47 of the distal end tip 41 are arranged in the inferior vena cava and the blood removal side hole 164 of the shaft portion 133 is arranged in the internal jugular vein.

As in the first embodiment, the expansion portion 32 is configured to have an inner diameter larger than that of the shaft portion 133. In a state where the through holes 46 and 47 and the blood removal side hole 164 are arranged in the blood removal targets, the expansion portion 32 is arranged in the inferior vena cava, which is a relatively thick blood vessel, and the shaft portion 133 is arranged in the femoral vein, which is a relatively thin blood vessel.

As illustrated in FIG. 11, a lock connector 136 includes a first lock connector 137 communicating with the first lumen 61 and a second lock connector 138 provided in parallel with the first lock connector 137 and communicating with the second lumen 62. The lock connector 136 is a Y-shaped Y connector formed by branching the first lock connector 137 from the second lock connector 138.

The first lock connector 137 is connected to a proximal end portion of the third tube 161. The second lock connector 138 is coaxially connected to a proximal end portion of the shaft portion 133. A blood supply tube (blood supply line) is connected to the first lock connector 137, and a blood removal tube (blood removal line) is connected to the second lock connector 138.

The intermediate portion 34 exhibits the same function as that of the first embodiment and also has the same function and effect.

As described above, according to the catheter 60 according to the present embodiment, it is possible to perform both functions of blood removal and blood supply with one catheter.

Although the catheter according to the present invention has been described through the embodiments, the present invention is not limited to only the configuration described in the embodiments and the modifications, and can be appropriately changed based on the description of the claims.

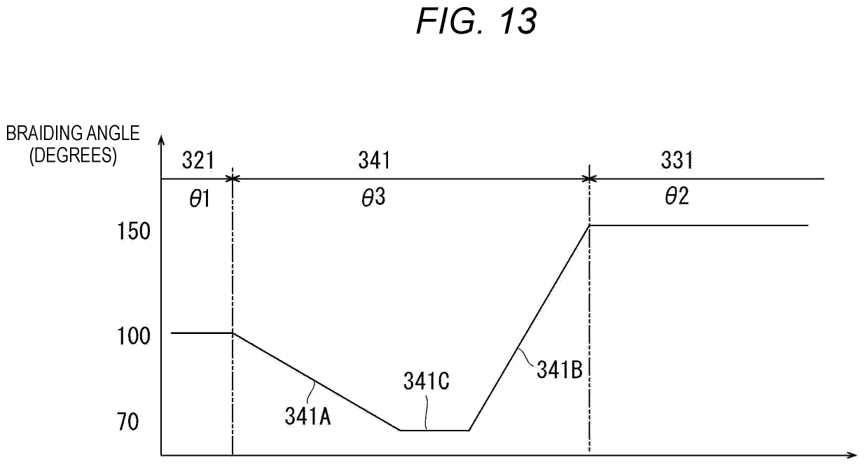

For example, in the first embodiment described above, as illustrated in FIG. 6, a third reinforcing body 341 has a first region 341A in which a third braiding angle θ3 is configured to gradually decrease from a first braiding angle θ1 of a first reinforcing body 321, and a second region 341B in which the third braiding angle θ3 is configured to gradually increase continuously from the first region 341A toward a second braiding angle θ2 of a second reinforcing body 331. However, as illustrated in FIG. 13, the third reinforcing body 341 may have a constant region 341C where the third braiding angle θ3 is constant between the first region 341A and the second region 341B. According to the catheter 30 configured as described above, a length along an axial direction of the intermediate portion 34 can be lengthened, and the catheter 30 can also be preferably applied to a catheter having a relatively long length along the axial direction.

Material constituting a wire W is not limited to a configuration in which the wire W is made of a shape memory material as long as the material has a restoring force of returning to the original shape by deformation and has a function of reinforcing a resin layer, and examples of the material can include a known elastic material.

In the second embodiment described above, the through holes 46 and 47 and the blood removal side hole 164 are used for blood removal, and the blood supply side hole 163 is used for blood supply. However, the through holes 46 and 47 and the side hole 164 may be used for blood supply, and the side hole 163 may be used for blood removal.

Further, in the first embodiment and the second embodiment described above, the expansion portion 32 includes the first resin layer 322, but is not limited to this configuration, and may not include the first resin layer.

What is claimed is:

1. A percutaneous catheter that extends in an axial direction and through which blood is passed, the percutaneous catheter comprising:
an expansion portion extending in the axial direction to be expandable and having a respective inner diameter and a respective outer diameter;
a shaft portion extending in the axial direction and provided on a proximal end side in an insertion direction of the expansion portion and having a respective inner diameter and a respective outer diameter; and
an intermediate portion provided between the expansion portion and the shaft portion, wherein:
the respective inner diameter and outer diameter of the expansion portion are respectively larger than the inner diameter and outer diameter of the shaft portion, and wherein the expansion portion is configured to have higher elasticity than that of the shaft portion;
the intermediate portion is configured to have a respective inner diameter and a respective outer diameter gradually decreasing from the expansion portion to the shaft portion;
the expansion portion comprises a first reinforcing body including first braided wires which intersect with each other in the first reinforcing body at a first braiding angle;
the shaft portion comprises a second reinforcing body including second braided wires which intersect with each other at a second braiding angle;
the intermediate portion includes a third reinforcing body including third braided wires which intersect with each other at a third braiding angle; and
the third reinforcing body is configured such that the third braiding angle is smaller than the first braiding angle and the second braiding angle.

2. The percutaneous catheter according to claim 1, wherein the third reinforcing body has a first region in which the third braiding angle is configured to gradually decrease from the first braiding angle of the first reinforcing body, and a second region in which the third braiding angle is configured to gradually increase continuously from the braiding angle of the first region toward the second braiding angle of the second reinforcing body.

3. The percutaneous catheter according to claim 1, wherein the third reinforcing body includes a constant region where the third braiding angle is constant.

4. The percutaneous catheter according to claim 1, wherein:
the first braiding angle of the first reinforcing body is in a range of 100 degrees to 120 degrees;
the second braiding angle of the second reinforcing body is in a range of 130 degrees to 150 degrees; and
the third braiding angle of the third reinforcing body is equal to or larger than 60 degrees.

5. A percutaneous catheter system for insertion into a blood vessel in an axial direction and through which blood flows, the percutaneous catheter system comprising:
a tubular body with a lumen for carrying the blood;
a tip at a distal end of the tubular body with a through hole; and
a stylet insertable through the tubular body to press against the tip to axially expand the tubular body;
wherein the tubular body comprises an expansion portion, a shaft portion, and an intermediate portion;
wherein the expansion portion extends in the axial direction to be expandable and has a respective inner diameter and a respective outer diameter;
wherein the shaft portion extends in the axial direction and is provided on a proximal end side in an insertion direction of the expansion portion and has a respective inner diameter and a respective outer diameter;
where the intermediate portion provided between the expansion portion and the shaft portion;
wherein the respective inner diameter and outer diameter of the expansion portion are respectively larger than the inner diameter and outer diameter of the shaft portion;
wherein the expansion portion is configured to have higher elasticity than an elasticity of the shaft portion;
wherein the intermediate portion is configured to have a respective inner diameter and a respective outer diameter gradually decreasing from the expansion portion to the shaft portion;
wherein the expansion portion comprises a first reinforcing body including first braided wires which intersect with each other in the first reinforcing body at a first braiding angle;
wherein the shaft portion comprises a second reinforcing body including second braided wires which intersect with each other at a second braiding angle;
wherein the intermediate portion includes a third reinforcing body including third braided wires which intersect with each other at a third braiding angle; and
wherein at least a major portion of the third reinforcing body is configured such that the third braiding angle is smaller than the first braiding angle and the second braiding angle.

6. The percutaneous catheter system according to claim 5, wherein the third reinforcing body has a first region in which the third braiding angle is configured to gradually decrease from the first braiding angle of the first reinforcing body, and a second region in which the third braiding angle is configured to gradually increase continuously from the braiding angle of the first region toward the second braiding angle of the second reinforcing body.

7. The percutaneous catheter system according to claim 5, wherein the third reinforcing body includes a constant region where the third braiding angle is constant.

8. The percutaneous catheter system according to claim 5, wherein:
the first braiding angle of the first reinforcing body is in a range of 100 degrees to 120 degrees;
the second braiding angle of the second reinforcing body is in a range of 130 degrees to 150 degrees; and
the third braiding angle of the third reinforcing body is equal to or larger than 60 degrees.
